(19) United States
(12) Patent Application Publication
(10) Pub. No.: US 2011/0124984 A1
(43) Pub. Date: May 26, 2011

(54) Device for Aliquoting and Filtering Blood

(76) Inventor: Hervé Rostaing, Le Versoud (FR)

(21) Appl. No.: 12/935,632

(22) PCT Filed: Mar. 24, 2009
(86) PCT No.: PCT/FR09/00321

§ 371 Date: Dec. 1, 2010

(30) Foreign Application Priority Data
Mar. 31, 2008 (FR) 0801776

(51) Int. Cl. A61B 5/157; B01L 3/02

(52) U.S. Cl. 600/309; 422/513

(57) ABSTRACT

Device for aliquoting and filtering blood, comprising: an element (1, 10) for collecting blood (S); a capillary conduit (2) which has a capillary force greater than that of said collecting element and is in fluid communication therewith; and a dispensing element (3) which has a capillary force greater than that of the capillary conduit and is in fluid communication therewith; characterized in that said dispensing element comprises a filtering device (3) for separating blood cells from plasma.

The invention relates to a device for aliquoting and filtering blood. More particularly, the device of the invention makes it possible to aliquot, to filter, and to dispense blood in small quantities (microliters).

Numerous biomedical analysis techniques require a small quantity of blood to be filtered in order to prepare the plasma for analysis of the blood cells (essentially red and white corpuscles). A known method of performing such filtering consists in filling a container (such as a pipette cone or a capillary) of a given volume with whole blood, and then dispensing the content onto a filter in order to separate and recover the plasma separated from the solid components of the blood. Two distinct steps are therefore necessary, or indeed three if the step of taking the blood from the patient's body is also counted.

KABE Labortechnik sells microtubes having a capillary duct of calibrated volume projecting from the outside thereof (see a brochure at the Internet address: http://www.kabe-labortechnik.de/download/kapillarblut_en. pdf). In order to take a determined quantity of blood (of the order of 100 microliters (μL)) using such a device, and to place it on a filter, the procedure is as follows. Firstly a patient's finger is pricked with a lancet, and a drop of blood is extracted; thereafter the drop is brought up to the end of the capillary duct while it is held in a horizontal position. The tube fills progressively as a function of its capillary flow rate. Once filled, the microtube is put into a vertical position to empty the capillary duct onto a filter, emptying being assisted by tapping or vibration.

The operation is relatively complex and does not avoid all risks of contamination of or by the blood. Furthermore, capillary forces retain about 25% of the liquid in the duct, thereby affecting the accuracy of aliquoting.

It is also possible to replace the microtube with a pipette bulb so as to enable the capillary duct to be emptied by applying pressure. That solution enables emptying to be more complete and thus makes aliquoting more accurate, but the other drawbacks of the device remain.

The invention seeks to solve at least some of the drawbacks of the prior art.

The device of the invention enables blood to be aliquoted accurately and enables it to be dispensed substantially completely onto a filter, in a manner that takes place automatically, by capillarity.

In accordance with the invention, these results are obtained by a device for aliquoting and filtering blood in accordance with claim 1. Such a device comprises a collector element for collecting blood; a capillary duct presenting a capillary force stronger than that of said collector element and in fluid flow communication therewith; and a dispenser element presenting a capillary force stronger than that of the capillary duct and in fluid communication therewith; said dispenser element including a filter device for separating blood corpuscles from plasma.

Particular embodiments of the invention constitute the subject matter of dependent claims.

In a preferred embodiment of the invention, the aliquoting, dispensing, and filtering functions, and possibly also the sample-taking function, may be performed by a single plane device of structure that is very simple.

Various applications for the device of the invention may be envisaged.

In a first application, said filter device may be adapted to saturate with corpuscles after filtering a volume of blood that depends on the hematocrit content of said blood, and that is in any event less than the content of said collector element and of said capillary duct; visible marks are provided making it possible merely by inspection to estimate the volume of liquid present in said capillary duct. In this way, the hematocrit content of said blood may be determined from the estimated volume of blood remaining in the capillary duct after the filter device has saturated.

In a second application of the invention, the device may also include, upstream from said filter device, a detachable element of material that absorbs blood plasma and that, on being dried, is suitable for conserving the proteins contained in said plasma. In this way, a sample of plasma may be taken directly by the patient, at home. After being separated from the device and dried, the absorbent element may be sent by post to a laboratory in order to analyze the plasma proteins that are conserved therein.

Other characteristics, details, and advantages of the invention appear on reading the description made with reference to the accompanying drawings given by way of example and in which:

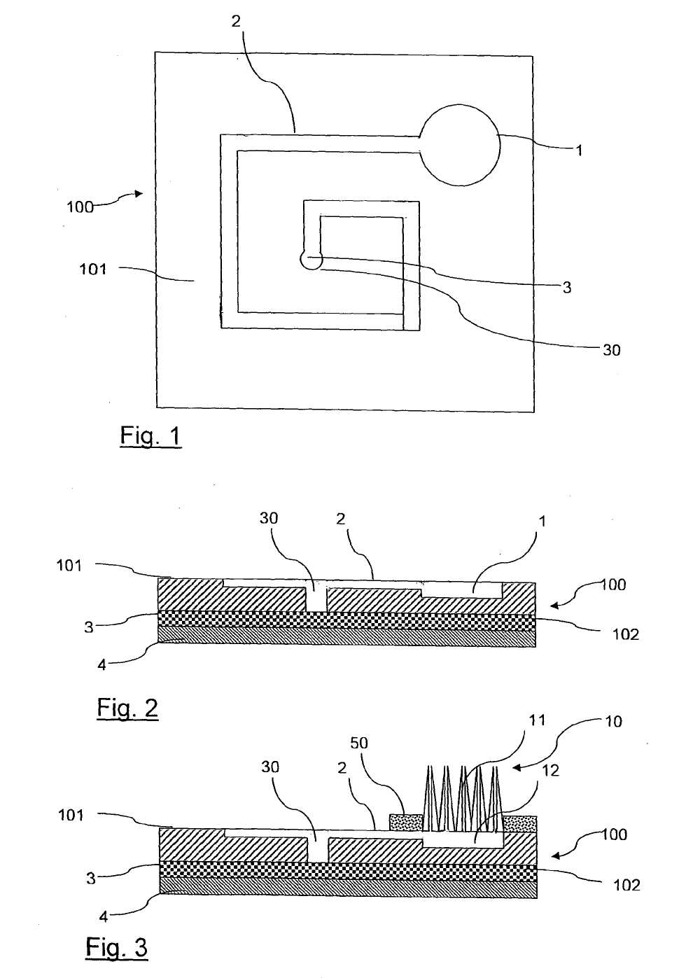

FIGS. 1 and 2 are a plan view and a section view of the blood aliquoting and dispensing section of a device in a first embodiment of the invention;

FIG. 3 is a section view of the blood aliquoting and dispensing section of a device in a second embodiment of the invention;

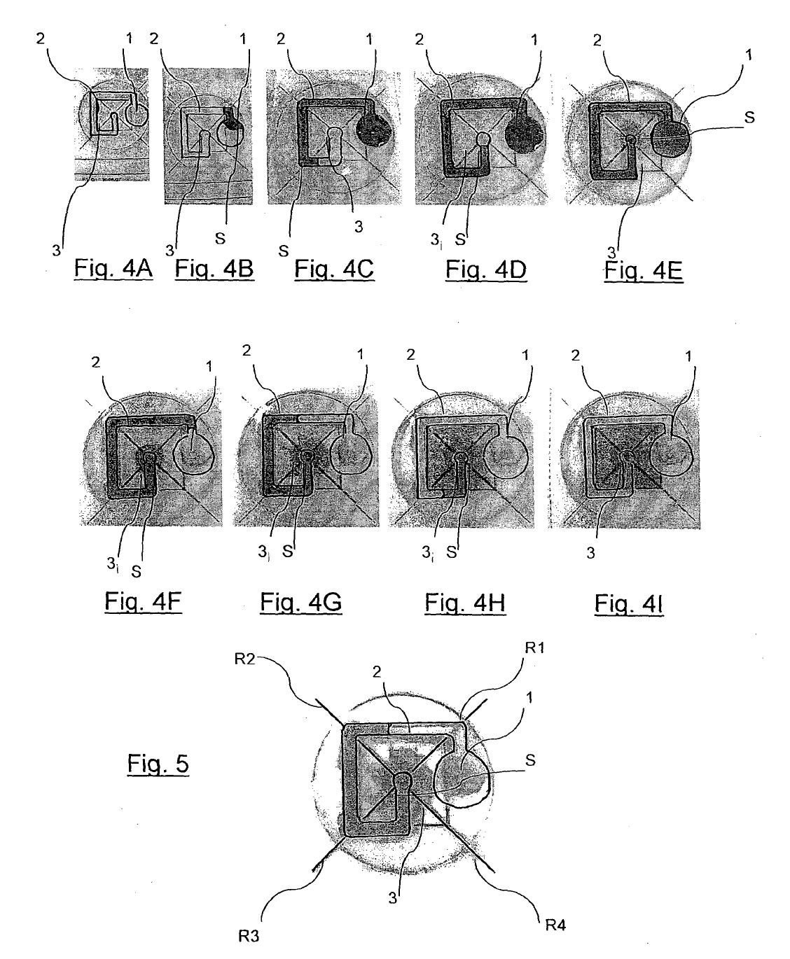

FIGS. 4A to 4E show the filling of the blood aliquoting and dispensing section of FIGS. 1 and 2;

FIGS. 4F to 4I show the emptying of the same section; and

FIG. 5 shows the application of a device of the invention to measuring the hematocrit content of a blood sample.

The blood aliquoting and dispensing section of FIGS. 1 and 2 presents a plane structure and it is made, e.g. by molding or machining, from an element 100 of hard or flexible plastics material. A soft plastic such as polydimethyl siloxane (PDMS) or a silicone is particularly preferred for implementing the invention as is a moldable or machineable plastics material that is transparent and preferably hydrophilic.

The following are formed on a surface of the element 100 that is referred to as its “top” surface 101: a well or depression 1 for collecting whole blood that is to be aliquoted and filtered; and a closed capillary duct 2 in fluid flow communication with the well 1. A cover needs to be placed over the duct 2.

At its end remote from the well 1, the duct 2 opens out into a hole 30 passing through the element 100. Against the surface of said element that is referred to as its “bottom” surface 102, there is a filter membrane 3, typically a paper membrane.

Beneath the paper membrane there may be an absorbent element 4 for performing a function that is explained below.

The internal volume of the collector element 1 and of the capillary duct taken together determines the quantity of blood that can be dispensed. This volume generally lies in the range 10 μL to 1 milliliter (mL), and preferably in the range 50 μL to 500 μL. In order to optimize aliquoting accuracy, it is preferable for the volume of the duct 2 to be considerably greater than that of the well 1: to achieve this said duct may be wound as a spiral or arranged as a zigzag. Overall, the device has dimensions of centimeter order. More precisely, the collector well 1 may have a diameter of the order 1 millimeter (mm) to 10 mm, and the capillary duct may present a section having an area of a few square millimeters (mm²) and a length of several centimeters (cm); for example, it is possible to envisage a duct having a rectangular section of 0.5 mm×2 mm for a length of 7.5 cm, giving an internal volume of 75 μL.

Prior to use, the capillary duct 2 and/or the well 1 may contain a variety of compounds in liquid, gel, or dry form such as anticoagulating agents for blood such as ethylene-diamine-tetra acetic acid (EDTA), globule agglutinating agents such as lectin, or agents that give rise to coagulation such prothrombin. It is particularly advantageous for the inside walls of the duct 2 to be coated in a gel that makes said walls hydrophilic and that progressively releases an anticoagulant while the capillary is being filled so as to prevent any start of coagulation.

As shown in FIGS. 4A to 4E, droplets of blood S may be deposited one by one in the well 1, so as to fill it progressively. Once the well 1 is filled sufficiently, the blood S begins to penetrate into the duct 2 into which it is sucked by capillarity. When the duct 2 is completely filled, the blood S reaches the filter membrane 3 via the hole 30 and it is sucked by the membrane, which presents a capillary force that is considerably stronger than that of the duct 2. At this point, drops are no longer deposited in the well 1. Since the volume of the well 1 plus the volume of the duct 2 are calibrated, the quantity of liquid inside the device is known accurately: this constitutes aliquoting.

The blood S continues to be sucked in by the filter membrane 3 until the device has been emptied completely (FIG. 4F to 4I): the aliquoted quantity of blood is thus dispensed.

The filter membrane 3 is adapted to retain blood cells while allowing plasma to pass. The plasma is thus collected downstream from said membrane.

In particular, the blood plasma may be collected by an absorbent element 4 in contact with the filter membrane 3 and detachable from the device. The absorbent element 4 soaks up the plasma and is then separated and dried. At this point it may be sent to a laboratory for biochemical analysis of the proteins that remain stored therein: this application is described in detail in French patent application FR 07/07709 filed on Nov. 2, 2007.

In another embodiment of the invention, the filter membrane 3 may be of dimensions so as to be saturated in blood corpuscles before the device is emptied completely. The exact quantity of blood that can pass through the filter before all of its pores become obstructed by blood cells (red and white corpuscles, platelets), depends on its hematocrit content. This content may thus be estimated by measuring the quantity of liquid that remains inside the device after it has been partially emptied. This measurement may be performed approximately using visible marks (lines R1 to R4 in FIG. 5) that are provided on the device. It can be understood that under such circumstances the plane element 100 must be transparent.

In yet another embodiment, the plasma may be collected in a vessel located downstream from the filter 3.

An array of micropillars may also constitute a dispenser element suitable for implementing the invention. For example, these may be silicon micropillars with a square section of 50 micrometers (μm)×50 μm, at a separation spacing likewise equal to 50 μm and having a height of 200 μm. These micropillars may be coated in appropriate chemical substances (e.g. antibody-antigen binding, streptavidin) for performing pretreatment on blood, e.g. such as capturing analytes (RNA, proteins) that can subsequently be detected through the device or collected for analysis.

FIG. 3 shows a particularly advantageous variant of the device of the invention having a collector element 10 constituted by an array of microneedles 11, preferably hollow microneedles, and by a reservoir 12 incorporated in the plane element 100 and located beneath said array.

The microneedles 11 are adapted to pass through a patient's skin, e.g. at the end of a finger, and to extract a small quantity of blood; they therefore need to have a length of at least 200 μm, and preferably of 1 mm to 2 mm in order to penetrate into the dermis. This minimizes any risk of infection for the patient and any risk of contamination for the sample taken. In addition, the blood is extracted, aliquoted, and dispensed in a single operation.

Preferably, an adhesive bonding region 50, e.g. in the form of a ring, completely surrounds the collector element. When taking a blood sample, the patient presses a finger onto said collector element, possibly after pricking the finger with a lancet (if the device does not have microneedles). The adhesive region 50 serves to form a sealed connection between the finger and the device so as to prevent any dispersion of blood and the associated biological risks and also prevent any prolonged contact between blood and air that would be likely to accelerate coagulation. The patient keeps the finger on the device throughout the filling stage. Once the duct 2 is fill, the patient takes the finger away, thereby enabling the device to empty quickly by capillarity: so long as the finger is present, emptying is slowed down and limited to the rate at which blood flows, whereas once the finger has been removed the rate is controlled by the capillary force at the outlet from the device. The adhesive connection does not prevent the finger being massaged, which assists in extracting blood. The collector element may also include pillars that are more or less flexible and that assist in locally massaging the finger and thus facilitating the flow of blood from the finger into the device.

1. A device for aliquoting and filtering blood, the device comprising:
a collector element for collecting blood;
a capillary duct presenting a capillary force stronger than that of said collector element and in fluid flow communication therewith; and
a dispenser element presenting a capillary force stronger than that of the capillary duct and in fluid communication therewith;
the device being characterized in that the dispenser element includes a filter device for separating blood corpuscles from plasma.

2. A device according to claim 1, wherein said collector element is a well or depression.

3. A device according to claim 1, wherein said collector element includes an array of microneedles and a reservoir located beneath the array.

4. A device according to claim 1, wherein an adhesive zone is provided on a surface of said device in register with said collector element so as to provide leaktight contact between said collector element and a zone of a patient's skin.

5. A device according to claim 1, wherein said collector element and said capillary duct are adapted to contain a volume of blood lying in the range 10 μL to 1 mL, and preferably in the range 50 μL to 500 μL.

6. A device according to claim 1, including visible marks making it possible, merely by inspection, to estimate the volume of blood present in said capillary duct.

7. A device according to claim 1, wherein said collector element and/or said capillary duct contain at least one chemical substance selected from:
anticoagulating agents for blood;
agglutinating agents for blood corpuscles; and
coagulating agents for blood.

8. A device according to claim 7, wherein at least one of the inside walls of said capillary duct is coated in a hydrophilic gel that progressively releases a blood anticoagulating agent while the duct is being filled with blood for aliquoting and filtering.

9. A device according to claim 1, wherein said collector element and said capillary duct are integrated in a common element of plane structure.

10. A device according to claim 9, wherein said element of plane structure is transparent.

11. A device according to claim 9, wherein said element of plane structure is made by molding or machining a plastics material.

12. A device according to claim 1, wherein said collector element and said capillary duct are incorporated in a surface of a single element of plane structure, said filter device comprising a membrane placed on an opposite face of said element, the capillary duct and the filter device being put into fluid flow communication via a hole passing through the element.

13. A device according to claim 1, wherein said filter device is a paper filter.

14. A device according to claim 1, wherein said filter device is adapted to become saturated in corpuscles after filtering a volume of blood that depends on the hematocrit content of said blood and that is in any event less than the content of said collector element plus said capillary duct; and wherein visible marks (R1-R4) are provided that make it possible, by mere inspection, to estimate the volume of liquid present in said capillary duct; thereby enabling the hematocrit content of said blood to be determined on the basis of an estimate of the volume of blood remaining in the capillary duct after the filter device has saturated.

15. A device according to claim 1, adapted to take blood samples and also including, downstream from said filter device, a detachable element of material that absorbs blood plasma and that, on being dried, is suitable for conserving the proteins contained in said plasma.
